(19) United States
(12) Patent Application Publication
(10) Pub. No.: US 2010/0262184 A1
(43) Pub. Date: Oct. 14, 2010

(54) BIO-ACTIVE CONSTRUCT CREATED BETWEEN FIXATION DEVICE AND SUTURE FIXED IN BONE

(76) Inventor: Peter J. Dreyfuss, Naples, FL (US)

Correspondence Address: DICKSTEIN SHAPIRO LLP, 1825 EYE STREET NW, Washington, DC, 20006-5403, US

(21) Appl. No.: 12/725,279

(22) Filed: Mar. 16, 2010

Related U.S. Application Data
(60) Provisional application No. 61/167,801, filed on Apr. 8, 2009.

(51) Int. Cl. A61B 17/04

(52) U.S. Cl. 606/228; 606/232

(57) ABSTRACT

Methods and constructs including a fixation device and a suture for fixation of soft tissue to bone, or of soft tissue to soft tissue, which amplifies the body's healing response created by the introduction of the suture material and the material properties of the fixation device. Fixation of soft tissue to bone (or of soft tissue to soft tissue) is conducted using a suture (for example, a suture strand, braid, a suture tape, or a combination thereof) and a fixation device (for example, a bone anchor, implant or screw). The suture and fixation device are manufactured from materials that have properties to amplify the body's healing response. Materials such as synthetic bioresorbable polymers (for example, poly-lactic acid) are utilized in the fabrication of orthopedic fixation devices. Once these materials are introduced into the body and are exposed to in vivo conditions, the devices manufactured with these materials undergo hydrolysis and degrade while maintaining specific mechanical properties over time.

### CROSS-REFERENCE TO RELATED APPLICATIONS

This application claims the benefit of U.S. Provisional Application No. 61/167,801, filed Apr. 8, 2009, the entire disclosure of which is incorporated by reference herein.

### FIELD OF THE INVENTION

The present invention relates to the field of surgery and, more particularly, to a device and method for fixation of soft tissue to bone, or of soft tissue to soft tissue, by amplifying the healing response created by the introduction of a suture material and the material properties of a fixation device.

### BACKGROUND OF THE INVENTION

Fixation of soft tissue to bone, or of soft tissue to soft tissue, typically involves the formation of an incision to access the surgical site and then reattachment of the soft tissue. When soft tissue is attached to bone, the surgeon drills a cavity in the bone and inserts a fixation device such as a screw or a bone anchor. Typically, the bone screw or anchor is formed of metal, composite, plastic or bioabsorbable material, and is held in place by threads or by barbs. If an anchor is employed, the anchor typically includes an eyelet through which a suture is threaded. After placing the anchor, the surgeon ties the suture through the soft tissue, connecting it to the eyelet of the bone anchor, thus re-approximating the soft tissue to the bone. The technique is repeated multiple times at different locations in the bone, with a separate knot tied at each location. If multiple sutures are used, however, regrowth of the soft tissue during natural healing is difficult as a result of the multiple suture knots. In addition, multiple sutures attached to the fixation devices (such as anchors or screws) increase the risk of suture slippage associated with these devices.

### SUMMARY OF THE INVENTION

The present invention provides a method and a construct including a fixation device and a suture for fixation of soft tissue to bone, or of soft tissue to soft tissue, which amplifies the body's healing response created by the introduction of the suture material and the material properties of the fixation device.

Fixation of soft tissue to bone (or of soft tissue to soft tissue) is performed using a suture (for example, a suture strand, braid, a suture tape, or a combination thereof) and a fixation device (for example, a bone anchor, implant or screw). The suture and fixation device are manufactured from materials that have properties to amplify the body's healing response. Materials such as synthetic bioresorbable polymers (for example, poly-lactic acid) are utilized in the fabrication of orthopedic fixation devices. Once these materials are introduced into the body and are exposed to in vivo conditions, the devices manufactured with these materials undergo hydrolysis and degrade while maintaining specific mechanical properties over time. To improve the properties of the polymeric biomaterial, ceramic materials (for example: tri-calcium phosphate, hydroxyapatite, or calcium phosphate) are added to the polymer construct for achieving a degradable and osteoconductive implant. Osteoconductivity is defined as the ability to support bone ingrowth by providing a structure into which bone cells can migrate. A bioactive construct can then be achieved by using a suture manufactured with a material such as collagen with the fixation device. Collagen inherently has specific cell interaction peptides which are beneficial to cell seeding and cell attachment. Thus, by combining different variables (bioabsorbable polymers or composite/polymer bioabsorbable materials, and collagen-based materials), a bioactive construct including a fixation device and a suture can be created which can amplify the healing response. This healing response begins to occur after the fixation device and suture have been implanted into the bone. The proximity of the suture on the outside of the fixation device and the bone in combination with the material properties of the suture and the fixation device facilitates tissue/bone growth through the suture and the fixation device forming a stronger construct than what was initially implanted.

In an exemplary and illustrative embodiment only, the suture is manufactured from collagen (for example, stuffed with collagen or coated with collagen) and the fixation device is a bio-composite or bio-absorbable anchor.

These and other features and advantages of the present invention will become apparent from the following description of the invention that is provided in connection with the accompanying drawing and illustrated embodiments of the invention.

### BRIEF DESCRIPTION OF THE DRAWING

FIG. 1 illustrates a schematic view of a fixation device with an attached suture, for fixating soft tissue to bone, in accordance with an embodiment of the present invention.

### DETAILED DESCRIPTION OF THE PREFERRED EMBODIMENTS

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention. Accordingly, the invention is not to be considered as limited by the foregoing description.

The present invention provides methods and constructs for fixation of soft tissue to bone (or of soft tissue to soft tissue) which amplify the healing response created by the introduction of a suture material and the material properties of a fixation device, resulting in the elimination of suture slippage associated with the fixation device. As detailed below, fixation of soft tissue to bone is performed using a suture (for example, a suture strand, braid, suture tape, or a combination thereof) and a fixation device (for example, a bone anchor, implant or screw) wherein the material properties of the suture and the fixation device amplify the healing response when implanted in vivo.

Selection of the suture material and of the fixation device material depends upon the compatibility of the materials and the characteristics of the surgical procedure (for example, the osteoconductive nature of the bone, the number of the fixation devices necessary to accomplish the reattachment, and the extent of the interference fit between the suture material and the material of the fixation device, among others). In an exemplary and illustrative embodiment only, the suture comprises collagen and the fixation device is a bio-composite or bio-absorbable anchor. In an exemplary and illustrative embodiment only, the suture is a collagen tape or a collagen stuffed suture, and the fixation device is a bio-composite or bio-absorbable anchor.

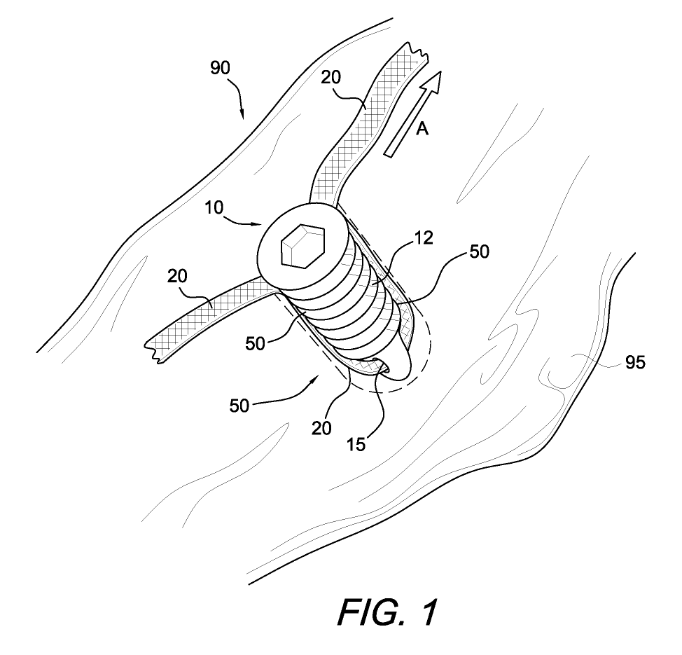

FIG. 1 illustrates a schematic view of a surgical site 90 undergoing a method of fixation of soft tissue to bone (or of soft tissue to soft tissue) by the method of the present invention. A fixation device 10 is secured within a cavity, tunnel or hole formed within bone 95. Fixation device 10 may be an anchor, screw or implant, for example. In an exemplary embodiment only, fixation device 10 is a bio-composite or bio-absorbable anchor. As illustrated in FIG. 1, fixation device 10 includes a body 12 and an eyelet or opening 15 that allows a suture 20 (for example, a suture strand, braid or suture tape 20) to pass therethrough. Fixation device 10 may be provided, however, without an eyelet or opening so that the suture or suture tape does not run through an eyelet and, thus, will not be damaged by friction between the suture and the eyelet.

In an exemplary embodiment, suture 20 is secured to the eyelet 15 of the fixation device 10 and extends on the exterior of the body 12, as shown in FIG. 1. In an exemplary embodiment only, the suture 20 is a collagen suture or a collagen tape (for example, a collagen stuffed tape). When tension is applied to the suture tape 20 (in the direction of arrow A of FIG. 1, for example), the material of the suture 20 (i.e, the collagen) comes into contact with the material of the fixation device 10 and autologous tissue (such as bone) and creates a biologically “active” site 50 which induces a quicker healing response. Over time, active areas 50 provide increased fixation of the suture (and of tissue attached to suture) and eliminate slippage of the suture from within the cavity, tunnel or hole formed within bone 95. In addition, because of the elimination of suture slippage, the fixation device may be provided without a suture eyelet, eliminating therefore any friction between the suture and the eyelet.

In an exemplary embodiment only, fixation device 10 is a knotless fixation device 10 such as an Arthrex “PushLock” C anchor (as disclosed and described in U.S. Pat. No. 7,329,272, the disclosure of which is hereby incorporated by reference in its entirety, and as shown in FIG. 1) or an Arthrex “SwiveLock” C anchor (as disclosed and described in U.S. Patent Application Publication No. 2007/0191849, the disclosure of which is hereby incorporated by reference in its entirety).

The suture 20 of the present invention may be employed for various soft tissue to bone repairs (that employ, for example, at least one knotless fixation device). According to an exemplary embodiment only, the suture 20 of the present invention may be employed in a method for double row fixation of tendon to bone, as detailed in U.S. Patent Application Publication No. 2007/0191849. After passing the suture 20 through tissue, the respective ends of suture 20 are threaded through respective eyelets of the fixation device 10 (SwiveLock anchor 10). A driver (with a screw inserted on a rod of the driver) is advanced to the edge of a pilot hole and used to install the anchor 10 and the screw within the pilot hole to form a final construct.

The suture 20 of the present invention may contain collagen and/or strands of a high strength suture material with surgically-useful qualities, including knot tie down characteristics and handling, such as Arthrex FiberWire® suture disclosed in U.S. Pat. No. 6,716,234, the disclosure of which is incorporated herein by reference. The suture 20 of the present invention may be also a suture with biological material, as described in U.S. patent application Ser. No. 12/397,236, filed on Mar. 3, 2009, the disclosure of which is hereby incorporated by reference herein. The suture may be provided with optional colored strands (preferably black) to assist surgeons in distinguishing between suture lengths with the trace and suture lengths without the trace.

The suture 20 may also contain a bioabsorbable material, such as PLLA or one of the other polylactides, for example, and/or may be formed of twisted fibers having strands of a contrasting color added to the braided threads, to make the suture more visible during surgical procedures. The colored strands, preferably, may be dyed filaments or strands.

The suture 20 of the present invention may be used in conjunction with a growth material which may be any solid, semi-solid, viscous, flowable, gel or elastic composition or mixture. The growth material may contain growth factors such as autogenous growth factors, for example platelet-rich plasma (PRP), autologous factors, for example, autologous-conditioned plasma (ACP), optionally in combination with hyaluronic acid (HY acid) and/or with a coagulant such as thrombin. The collagen suture 20 may be soaked, for example, in platelet-rich plasma (PRP) or autologous-conditioned so that, once the suture contacts the fixation device 10 to become inserted into the pilot hole, the collagen and the growth material fill in the void in the bone and promote rapid healing of the surgical site.

The term “growth factor” as used in the present application is intended to include all factors, such as proteinaceous factors, for example, which play a role in the induction or conduction of growth of bone, ligaments, cartilage or other tissues associated with bone or joints. In particular, these growth factors include bFGF, aFGF, EGF (epidermal growth factor), PDGF (platelet-derived growth factor), IGF (insulin-like growth factor), TGF-β. I through III, including the TGF-β. superfamily (BMP-1 through 12, GDF 1 through 12, dpp, 60A, BIP, OF).

Optionally, the growth material may comprise additional osteoconductive bone adhesives, calcium carbonate, fatty acids, lubricants, antiseptic chemicals and/or antibiotics. In this case, other solution excipients such as buffer salts, sugars, anti-oxidants and preservatives to maintain the bioactivity of the growth material and a proper pH of the growth material may be also employed. The additional lubricants and/or the antiseptic and/or the antibiotic will typically be present in the growth material in a predetermined concentration range, which will be dependent upon the particular bone site and application, as well as the specific activity of the antiseptic and/or the antibiotic.

The suture 20 and fixation device 10 of the present invention may be employed in surgical procedures such as rotator cuff repair, Achilles tendon repair, patellar tendon repair, ACL/PCL reconstruction, hip and shoulder reconstruction procedures, and replacement for suture used in or with suture anchors.

An exemplary surgical procedure for tendon repair employing the method and devices of the present invention is the SpeedBridge™ technique, developed by Arthrex, Inc., which uses a threaded swivel anchor (such as disclosed in U.S. Patent Publication No. 2008/0004659, the disclosure of which is herein incorporated by reference) combined with FiberTape® (disclosed in U.S. Patent Publication No. 2005/0192631), the disclosure of which is herein incorporated by reference) to create a quick and secure SutureBridge construct with no knots and only two suture passing steps.

While the present invention is described herein with reference to illustrative embodiments for particular applications, it should be understood that the invention is not limited thereto. Those having ordinary skill in the art and access to the teachings provided herein will recognize additional modifications, applications, embodiments and substitution of equivalents all fall within the scope of the invention. Accordingly, the invention is not to be considered as limited by the foregoing description.

What is claimed as new and desired to be protected by Letters Patent of the United States is:

1. A bio-active construct, comprising:
a fixation device;
a suture structure comprising a bioabsorbable biological material, the suture structure extending along at least one side of the fixation device; and
a bio-active site formed by direct contact and interaction of the bioabsorbable biological material of the suture structure and a material of the fixation device.

2. The bio-active construct of claim 1, wherein the suture structure extends along two longitudinal sides and one lateral side of the fixation device.

3. The bio-active construct of claim 1, wherein the fixation device is an implant or a suture anchor.

4. The bio-active construct of claim 1, wherein the fixation device is a knotless fixation device and the suture material is passed through an eyelet of the knotless fixation device.

5. The bio-active construct of claim 1, wherein the fixation device is a swivel anchor or a push-in anchor.

6. The bio-active construct of claim 1, wherein the fixation device comprises a bioresorbable polymer and a ceramic material.

7. The bio-active construct of claim 1, wherein the fixation device comprises a bioresorbable polymer and the suture structure is a suture tape.

8. The bio-active construct of claim 1, wherein the biological material is at least one of a biopolymer, protein, graft, bodily fluid, growth factor, antiseptic, antibiotic and hormone.

9. The bio-active construct of claim 1, wherein the biological material comprises at least one of blood, blood components, platelet-rich plasma, autologous conditioned plasma and bone marrow aspirate.

10. The bio-active construct of claim 1, wherein the biological material comprises collagen.

11. The bio-active construct of claim 1, wherein the biological material is provided in the form of strands or a matrix.

12. A method of increasing the healing response of repaired tissue, comprising:
providing a knotless fixation device in the vicinity of a surgical site, the knotless fixation device comprising a bioresorbable polymer;
passing a suture material comprising a bioabsorbable biological material through an eyelet of the knotless fixation device; and
inserting the knotless fixation device with the suture material in the surgical site, so that the suture material directly contacts and interacts with the bioresorbable polymer of the knotless fixation device.

13. The method of claim 12, wherein the suture material is a suture strand or a suture tape, or a combination of suture strand and suture tape.

14. The method of claim 12, wherein the bioresorbable polymer is poly-lactic acid and the bioabsorbable biological material is collagen.

15. The method of claim 12, wherein the bioabsorbable biological component is at least one of bodily fluid, growth factor, antiseptic, antibiotic and hormone.

16. The method of claim 12, wherein the surgical site is part of a shoulder, a knee, a hip, or an elbow.
